(19) United States
(12) Patent Application Publication
(10) Pub. No.: US 2009/0221894 A1
(43) Pub. Date: Sep. 3, 2009

(54) MINIMALLY INVASIVE VESSEL LOCATION

(76) Inventors: Helge Myklebust, Stavanger (NO); Bob Neumar, Wayne, PA (US); Joshua Lampe, Philadelphia, PA (US)

Correspondence Address: DORSEY & WHITNEY LLP;INTELLECTUAL PROPERTY DEPARTMENT, SUITE 3400, 1420 FIFTH AVENUE, SEATTLE, WA, 98101, US

(21) Appl. No.: 12/329,353

(22) Filed: Dec. 5, 2008

Related U.S. Application Data
(60) Provisional application No. 61/005,660, filed on Dec. 5, 2007.

(51) Int. Cl. A61B 5/04; A61M 25/09; A61B 5/05

(52) U.S. Cl. 600/373; 600/585; 600/407

(57) ABSTRACT

The present invention is related to a blood vessel locator and a method for locating blood vessels. In one or more embodiments, the locator comprises at least one filiform element, a sensor device and a display. The filiform elements are connected to the sensor device, which is arranged to detect a parameter indicative of the presence of a blood vessel and connected to a display, and the display is arranged to give an indication of vessel location.

### CROSS-REFERENCE TO RELATED APPLICATION

This application is based on and claims the benefit of U.S. Provisional Patent Application No. 61/005,660, filed Dec. 5, 2007.

The entire disclosure of the prior application is considered to be part of the disclosure of the instant application and is hereby incorporated by reference therein.

### TECHNICAL FIELD

This invention relates to devices and method for locating a blood vessel in a body.

### BACKGROUND OF THE INVENTION

One of the most frequently performed medical procedures is the insertion of a needle into a live human body for the purpose of drawing blood from a vessel, delivering fluids and drugs, inserting a catheter, performing diagnostic tests, etc. Despite the frequency of this procedure, accurate needle insertion is often challenging due to the difficulty in locating a desired blood vessel. Several factors confounding blood vessel location include low or no blood pressure, such as in elderly and cardiac arrest patients, small blood vessels, such as in children, or the fact that the vessels can not be visualized or palpated, such in obese patients or patients with tissue damage.

Several methods are used to locate blood vessels. One widespread technique used in clinical practice involves using anatomical landmarks to estimate the location of blood vessels based on a position of visible features, such as articulations and muscles, and palpation of non-visible structures. A clear disadvantage of this method is its low accuracy for certain patients, such as obese and elderly patients and certain medical situations, such as patients under cardiac arrest.

“Popping” detection is also a widespread method for vessel location. This technique comprises inserting a needle in a body part at the site where a vessel is supposed to be. Because a vessel wall is elastic up to a certain degree, it is possible to notice a change in mechanical resistance to penetration when the needle perforates the vessel wall. This method also has several disadvantages. First, this method can require several attempts. Second, the vessel walls in elderly, children and cardiac arrest patients usually lacks the elasticity necessary to ensure detection. Third, the use of gloves reduces the operator's sensation of popping as well as the ability to palpate non-visible structures.

“Flash back” observation is observation of blood in the introduced needle when a blood vessel is perforated. This method has similar disadvantages as “popping” detection.

U.S. Pat. No. 5,280,787 and U.S. Pat. No. 6,056,692 disclose ultrasonic scanning of a body part to locate blood vessels. This technique requires advanced equipment and is again subject to error due to reduced or non existent blood flow. Additionally, this method requires significant training to ensure proper use of the ultrasound device.

FR Patent No. 2448337 describes a device comprising several needles situated in parallel and connected to a common source of vacuum. The device is situated on the patient's skin and pressed down so that the needles penetrate the underlying tissue. When one needle pierces a blood vessel, blood will appear on the needle's end as a consequence of “flash back”. This device is based on the operator's ability to distinguish which needle is producing flash back.

As one can see, techniques with an acceptable accuracy, such as landmarks, flash-back, and popping, require trial and error and are performed by a single needle, which leads to delays, potential injury and patient discomfort and is generally inefficient.

Therefore, there is a need for a vessel locator which provides swift vessel location with minimal injury and discomfort.

### SUMMARY OF THE INVENTION

One aspect of the invention includes a locator comprising at least one sensor device, a display device, and at least one filiform element. The filiform element is coupled to the sensor device and the display device. The sensor device is operable to detect a parameter indicative of presence of a blood vessel. The display device is operable to give an indication of accurate needle placement and other feedback to a user or operator. The invention comprises also a method for locating a blood vessel comprising: detecting a parameter indicative of the presence of a blood vessel by means of a sensor device connected to at least one filiform element, and providing an indication of blood vessel location.

### BRIEF DESCRIPTION OF THE DRAWINGS

FIG. 1 is a schematic illustration of a locator according to one embodiment of the invention.

FIG. 2 is a schematic illustration of a locator in use according to one embodiment of the invention.

FIG. 3 is a schematic illustration of a filiform element according to one embodiment of the invention.

FIG. 4 is a schematic illustration of a filiform element according to one embodiment of the invention.

FIG. 5 shows use of the invention according to an embodiment of the invention as a guidance for placement of a needle.

FIG. 6 is a schematic illustration of a locator 1 according to another embodiment of the invention.

FIG. 7 is a schematic illustration of a locator 1 in use according to one embodiment of the invention.

FIG. 8 is a schematic illustration of a locator according to another embodiment of the invention.

FIG. 9 is a schematic illustration a locator 1 having an array of filiform elements according to another embodiment of the invention.

FIG. 10 is a schematic illustration of a display device of a locator according to some embodiments of the invention

FIG. 11 is a schematic illustration of a locator 1 according to another embodiment of the invention.

FIGS. 12 and 13 show impedance values for different body tissues.

### DETAILED DESCRIPTION OF PREFERRED EMBODIMENTS

Embodiments of the present invention are directed toward locating blood vessels in a body. One or more embodiments of the invention locate a blood vessel using the sensing device without the use of traditional needles, which often causes injury to tissue and vessels. Certain details are set forth below to provide a sufficient understanding of the embodiments of the invention. However, it will be clear to one skilled in the art that various embodiments of the invention may be practiced without these particular details.

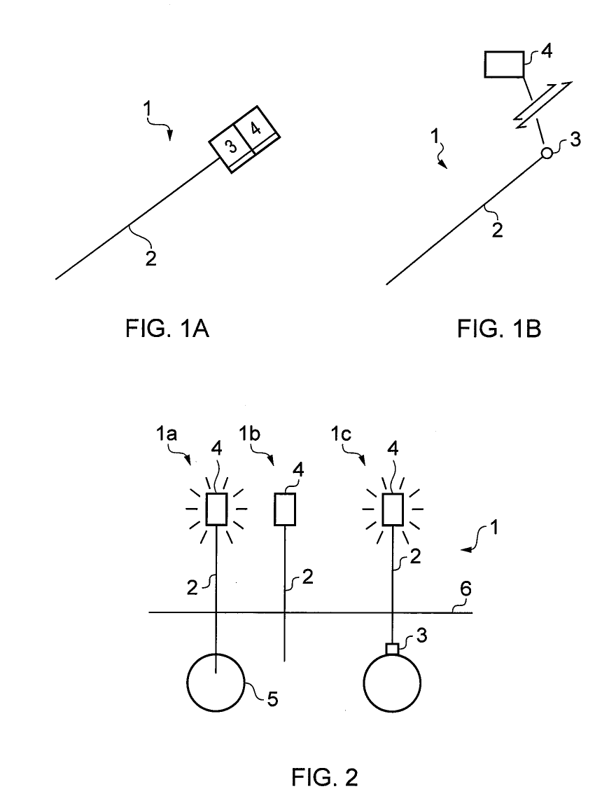

FIG. 1 is a schematic illustration of a locator 1 according to one embodiment of the invention. The locator 1 comprises a filiform element 2, a sensor device 3 and a display device 4. The filiform element 2 is operable to be introduced into a tissue overlying a blood vessel. In particular, the filiform element 2 is operable to penetrate into the tissue overlying a desired blood vessel until a part of a filiform element 2 is situated within the blood vessel. The sensor device 3 is operable to detect a parameter indicative of the presence of a blood vessel. The display device 4 is operable to indicate the presence of a blood vessel.

In one embodiment, the sensor device 3 is physically connected to the filiform element 2, such as by cables. In another embodiment, the sensor device is integral with the filiform element 2. In yet another embodiments the sensor device is coupled to the filiform element wirelessly.

In some embodiments, the display device 4 is integral with the locator 1 as is shown in FIG. 1A. In another embodiment, the display is a separate unit from the locator 1 as is shown in FIG. 1B. In the embodiment shown in FIG. 1B, the connection to the sensor device 3 from the display 4 may be implemented by cables or wirelessly.

The manner in which the display device 4 indicates a blood vessel location may take several forms. In one embodiment, the display device 4 provides a visual indication, such as changing color from red to green upon detection of a blood vessel. Another possible visual indication may include providing an image on the display device 4. The image may include the underlying tissue and the blood vessel once it is found. In another embodiment, the display device 4 provides an audio indication, such as emitting a sound when a blood vessel is detected. Another embodiment, may include amplifying the popping signal to give a tactile feedback. The desire to amplify the popping signal is based on the fact that a popping signal caused by a thin filiform element is weak and not easily detectable.

FIG. 2 is a schematic illustration of locators 1a, 1b, and 1c in use according to an embodiment of the invention. FIG. 2 shows skin surface 6 and blood vessels 5 underlying the skin surface 6. At locator 1a, a filiform element 2 has penetrated the blood vessel 5 and the display device 4 is activated. As will be known to person having ordinary skill in the art, a display device 4 can also be activated when no blood vessel is detected, such as to provide an indication that the device is turned on. At locator 1b, a filiform device 2 is introduced in the tissue but no indication of a blood vessel is given.

At locator 1c a filiform element 2 is in contact with the blood vessel 5 but has not penetrated the blood vessel and the display device 4 is activated. In this embodiment, the tip of the filiform element 2 comprises an impedance sensor 3. The impedance sensor 3 has an active volume around the tip and the tissue subject to the impedance measuring volume now characterizes the impedance value that can be measured. In this embodiment, the impedance sensor 3 recognizes the characteristic impedance of the vessel wall. The display device 4 is set to emit a visual element, such as a yellow signal, to indicate that the filiform element 2 is about to penetrate the wall of the blood vessel 5. In some embodiments, the sensor device 3 does not require physical contact with the blood vessel wall or the blood. For instance, a sensor 3 using optical methods, such as pulse oxymetry or a doppler ultrasound sensor may be used.

Although FIG. 2 shows the display device 4 being activated when situated directly on top of the blood vessels 5, as will be clear to a person having ordinary skill in the art and as mentioned above, it is possible to detect the proximity of a blood vessel from a distance from the sensor depending on the type of sensor being used. Therefore, it is possible to detect the proximity of a blood vessel around the filiform element's tip.

In some embodiments, it is also possible to provide marks on the filiform element 2 which indicate how deep the tip of the filiform element 2 is positioned in the tissue. This can be used to determine when a depth is reached in which a blood vessel should already have been found. In an alternative embodiment, a device is arranged such that the filiform element 2 is progressed automatically using a motor. In this case the progress of the filiform element 2 can be controlled by a separate sensor, for instance arranged as a roller potentiometer, where the progress of the filiform element turns a roller potentiometer, and a controller is arranged to translate potentiometer readings to millimeters. This embodiment will also give a depth indication similar to the marks discussed above.

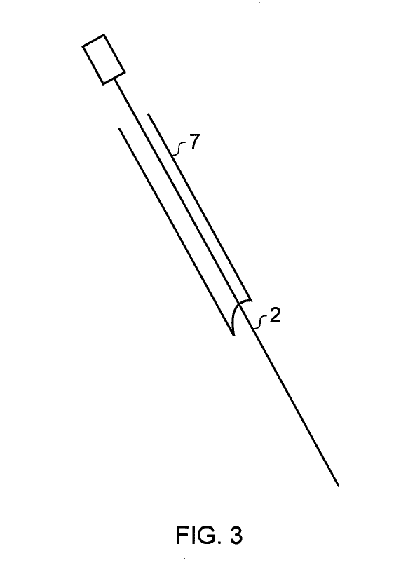

FIG. 3 is a schematic illustration of a filiform element 2 according to one embodiment of the invention. The filiform element 2 in FIG. 2 is a thin wire. In this embodiment, the filiform element 2 has a thickness that does not provide much rigidity, such as a filiform element 2 having a diameter of less than 0.4 mm. In such a case, a sleeve 7 can be used to provide rigidity to at least a portion of the filamentary element 2.

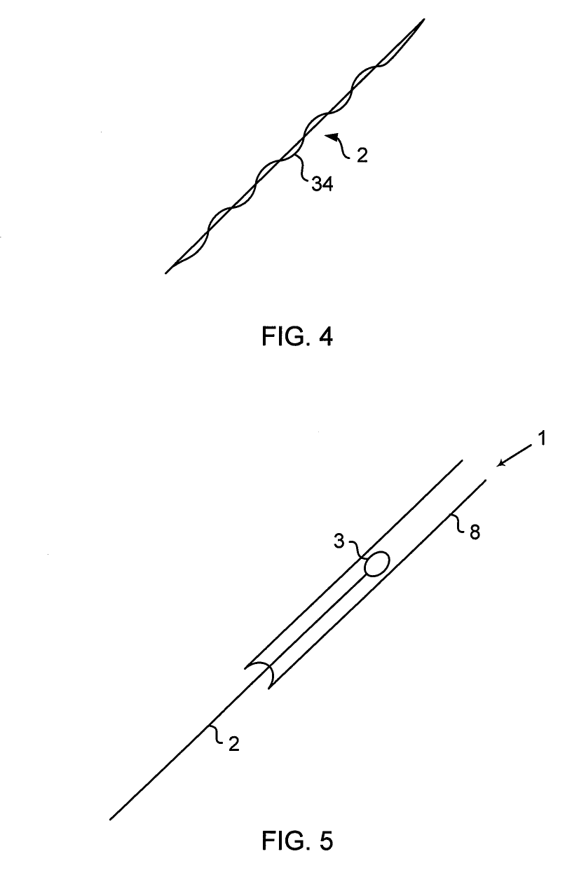

FIG. 4 is a schematic illustration of a filiform element 2 according to another embodiment of the invention. In this embodiment the filiform element 2 is provided with helicoidal protrusions 34, which facilitate insertion in the skin when rotating the element.

FIG. 5 is a schematic illustration of a locator 1 according to another embodiment of the invention. In this embodiment a sensor 3 and/or the display (not shown) have reduced dimensions so that a filamentary element 2 can be used as a guide wire for a hollow device, such as a catheter 8, needle, or cannula once a blood vessel is detected. In another embodiment the sensor 3 and/or the display can be releasably attached to the filiform element 2 so that the sensor 3 and/or the display can be removed once a blood vessel is located. In this embodiment, the filiform element 2 can act as a guide wire for the catheter 8, needle, or similar device.

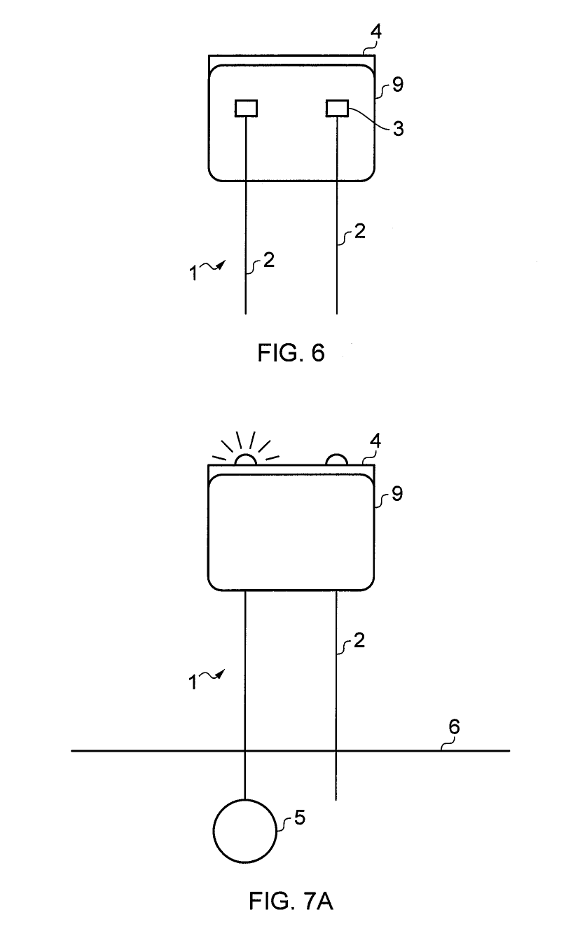

FIG. 6 is a schematic illustration of a locator 1 according to another embodiment of the invention. In this embodiment, the locator 1 comprises two filiform elements 2, two separate sensor devices 3 and at least one display device 4. In this embodiment each filiform element 2 is connected to a respective sensor 3. This embodiment can comprise a single display device or indicator for each sensor device 3 or one common display device showing a combination of signals from the sensors 3. The filiform elements 2 are arranged to be introduced into a tissue of a patient, where the tissue overlies a blood vessel. In this embodiment, locator 1 also comprises a body 9 which provides a mechanical coupling between the two filiform elements 2. In one embodiment, each of the filiform elements 2 are individual secured to the body 9. The mechanical coupling may be releasable or fixed. In another embodiment of the invention the filiform elements 2 are mechanically coupled to one another so that movement of one of the filiform elements 2 causes movement of the other filiform element 2. In the embodiment shown in FIG. 6 a single display device 4 is positioned on a surface of the body 9. In another embodiment, a number of display devices 4 are provided for a single filiform element 2 or for a group of filiform elements 2.

The filiform elements 2 may be of varying shape and material. This will allow the filiform elements 2 to be connected to a variety of differing sensor devices 3. In one embodiment, two similar sensor devices 3 are connected to non-similar filiform elements 2 to provide definition adjustment. This can be implemented by connecting two similar acceleration sensors to two filiform elements of different type, providing a coarse and a fine measurement or calibration measurement.

In some embodiments, the filiform element 2 may be implemented by means of an acupuncture needle. In one embodiment, the filiform element 2 is a thin needle with a diameter of about 0.2-1.0 mm and length of about 10-50 mm. A thin filiform element will not lead to injuries in a blood vessel, as is the case when a needle nicks a blood vessel without occluding the created opening. Typically, the filiform element 2 does not cause significant pain. Whether a filiform element 2 is hollow or not depends on the type of sensor device 3 attached to it.

The sensor device 3 may any sensor operable to detect a blood vessel. For instance, the sensor may be a pressure sensor, an impedance measuring device, a blood chemistry sensor, an acceleration sensor, a force sensor, a blood flow sensor, or a temperature sensor. When an acceleration or pressure sensor is used, the filiform element may be a thin wire. A pressure sensor will indicate presence of a blood vessel upon characteristics in the sensed pressure. With the pressure sensor in an artery, most pressures will be pulsatile with a mean pressure within 40-250 mmHg. With the pressure sensor in a vein, the pressure is non-pulsatile and has a mean value of 5-30 mmHg. During cardiac arrest, and with ongoing chest compressions, both pressures may be pulsatile with a mean value different from zero, typically around 60 mmHg. An impedance measuring device will sense the lower electrical impedance of blood when compared to other types of tissue. In this case the locator comprises one or more extra electrode(s) (separated from the filiform element or elements) to permit impedance measurement. Additionally, an impedance sensor would not require a hollow sensor, although in some embodiments a hollow sensor may be desired to allow for blood “flash-back”.

A blood chemistry sensor may be based on detecting specific substances present in blood. For instance, a blood sensor may detect blood due to the lower impedance of blood. An acceleration sensor connected to the filiform element will detect “popping” as a sudden change in acceleration as a filiform element enters trough a vessel wall. A blood flow sensor will detect fluid flow to determine the needle location. In physiological conditions, the flow rates in the arteries will be pulsatile and of a magnitude appropriate for vessel diameter and location eg 10 ml/s-400 ml/s. Under cardiac arrest conditions flow rates will depend upon quality of CPR, and may be as low a 0 ml/s to 100 ml/s. In the venous system blood flow will not be pulsatile, and flow rates are expected to be in lower ranges again dependent on vessel geometry and location, e.g., 0-100 ml/s. A blood temperature sensor may be used to determine accurate placement of a needle in a blood vessel. The temperature sensor may be active and provide heating to the blood to determine the heat transfer properties of the surrounding tissue, with the expectation that the conduction of heat in the blood stream will be better than the conduction of heat in the surrounding tissue. The blood temperature probe may be passive and measure the blood temperature. In these cases, the expected temperature will require calibration to the current treatment, such as with hypothermia. Additionally, the sensor may us optical means of detecting the blood vessel, such as pulse oxymetry or Doppler ultrasound sensors, In another embodiment, the sensor device 3 a small ultrasound doppler sensor, which detects blood because moving blood has a velocity. In another embodiment, the sensor device is an electrode, which reacts to the higher oxygen content of arterial blood.

In the embodiment that uses an impedance sensor for the sensing device 3, the filiform element 2 will work as an electrode and can thus be implemented with reduced dimensions. In some embodiments, the dimensions of the filiform element 2 is 0.15-1.0 mm in diameter and has a length of 10-50 mm. Stainless steel is a preferred material, but the needle may be electrically insulated except for the tip. Only the non-insulated portion will then form part of the impedance sensor. The length of the non-insulated part may depend on the type of vessel being located. For instance, if the vessel diameter is about 10 mm and the expected location is below about 25 mm of tissue, then the needle length should be at least 35 mm and the non-insulated length about 10 mm. The diameter is then chosen according to mechanical stability needed. In this case an additional electrode will be used for performing the measurement. The additional electrode does not need to be introduced in the tissue and can rest on the skin to form a closed loop for a current that is used to measure impedance.

The sensor device 3 may be coupled to the filiform element 2 in a variety of ways. In one embodiment, the sensor device 3 is assigned to a single filiform element 2 or a group of filiform elements 2. In the figures referenced above, the sensor devices 3 are shown as directly coupled to the filiform elements 2, but it is also possible to provide the sensor devices 3 coupled to filiform elements 2 by cables or other connectors and also wirelessly. As discussed above, the same applies to the connection between sensor the devices 3 and the display device 4.

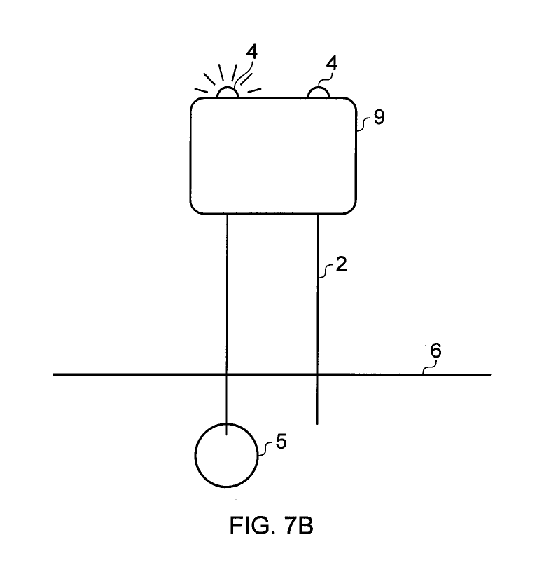

FIG. 7 is a schematic illustration of a locator 1 in use according to one embodiment of the invention. In particular, FIG. 7 shows a locator 1, skin surface 6 and a blood vessel 5 underlying the skin surface 6. FIG. 7A shows a filiform element 2 in contact with a detected blood vessel 5, but the filiform element has not penetrated the blood vessel. The corresponding visual element on the display device 4 is activated. The sensor device 3 (not shown) in this embodiment may be any type of appropriate sensor, such any of the pressure sensors mentioned above. This locator corresponds to the locator 1c illustrated in FIG. 2.

FIG. 7B shows a filiform element 2 that has penetrated blood vessel 5 and the corresponding part of the display device 4 is activated. The sensor device 3 in this embodiment may be a blood sensor. This locator corresponds to the locator 1a illustrated in FIG. 2.

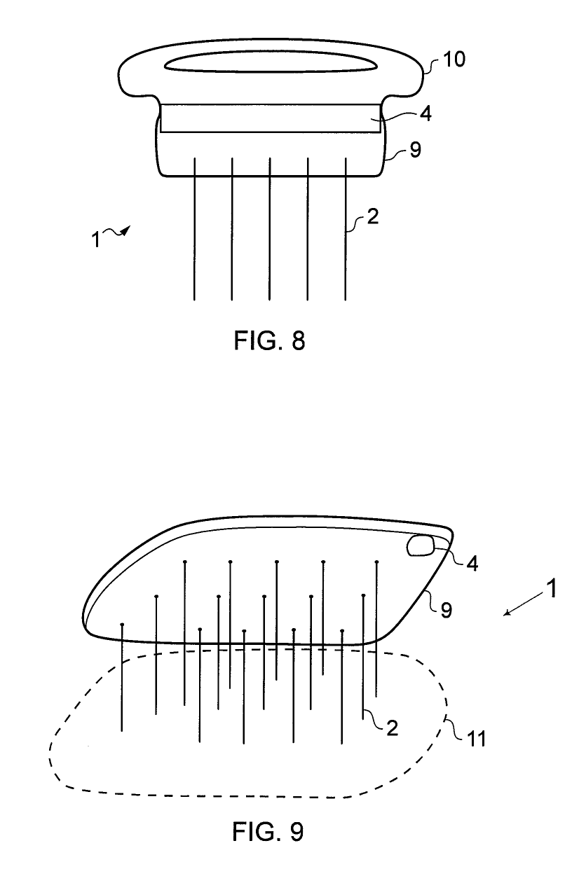

FIG. 8 is a schematic illustration of a locator according to another embodiment of the invention. In this embodiment, the locator 1 includes a single row of filiform elements 2. The use of two or more filiform elements 2 leads to an improved detection with fewer attempts. The time it takes to locate a blood vessel when using multiple filiform elements that are introduced in parallel may be significantly reduced over the prior art method of using a trial and error method with a single needle.

When several filiform elements are used, these can be connected to a common holder. In the embodiment shown in FIG. 8, the locator 2 comprises a holder 10. The holder 10 may be integral with a body 9. The holder 10 can comprise a display device 4 or the display device 4 may be arranged on the body 9. As mentioned above, it is also possible to provide a separate display device 4. The display device may be coupled to the body 9 or the holder 10 wirelessly or by cables. The holder 10 can also be formed as a knob or have any other design which facilitates controlled manual positioning on the detection area. In another embodiment, the holder 10 can also include the display device, which as indicated earlier may include a light source, a sound emitting device, an LCD display to enable communication with the operator, for instance providing information relating to battery status, technical status.

FIG. 9 illustrates a locator 1 according to another embodiment of the invention. The locator 1 in this embodiment includes an array of elements 2 to facilitate location of blood vessels in an enlarged area. The filiform elements 2 cover a detection area 11 and can locate a blood vessel within this area.

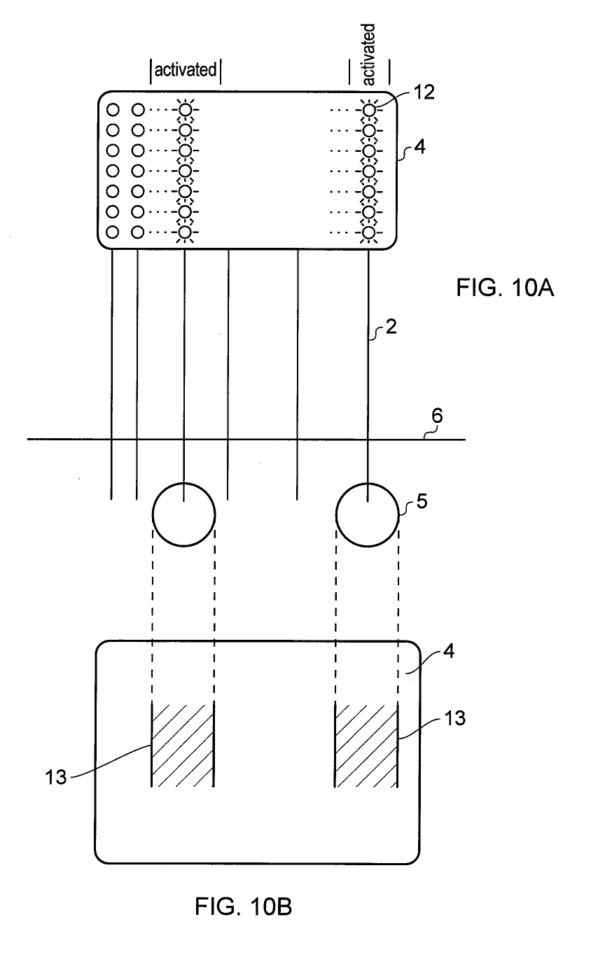

FIG. 10 is a schematic illustration of a display device of a locator according to some embodiments of the invention. For instance, FIG. 10A shows an embodiment of the invention that may be used in conjunction with the locator shown in FIG. 9. A display device 4 comprises several LCD segments or LEDs 12, where each LCD segment or LED is assigned a single filiform element 2 or a group of filiform elements 2. Activation of the LCD segment or LEDs will be controlled by means of a processor (not shown) in response to signals received from the sensors (not shown). Although the display device 4 with several LCD segments or LEDs is shown in relation with an array of filiform elements, it is also possible to use it in connection with a single row of filiform elements as in the locator 1 shown in FIG. 8.

FIG. 10B shows a display device 4 showing an area 13 where a blood vessel is situated. The display device 4 may be an LCD or LED display device. Similarly to the display device in FIG. 10A, the LCD display in FIG. 10B can be used for a locator comprising a single row of filiform elements. As discussed above, the display devices shown in FIGS. 10A and 10B may be integrated with the locator or provided separately.

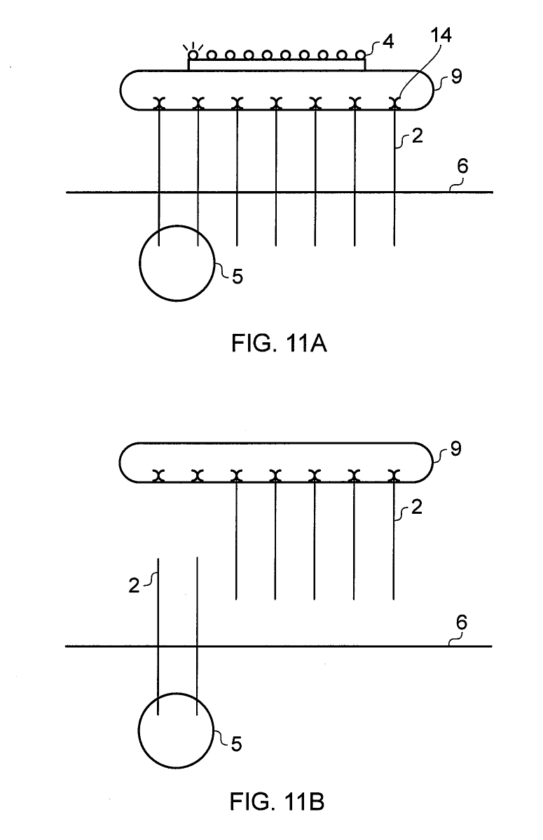

FIG. 11 is a schematic illustration of a locator 1 according to another embodiment of the invention. In this embodiment releasable fixation devices 14 are provided in the body 9. FIG. 11A shows the locator 1 introduced to a detection area. FIG. 11B shows two fixation devices 14a and 14b released and two corresponding filiform elements 2 so that the filiform elements 2 remain in the blood vessel 5 after the locator 1 is removed. Thus, it is possible to use the two filiform elements 2 that remain in the blood vessel 5 as guidewires or to introduce a needle in the corresponding locations independently of the elements. The display device provides the location of the blood vessel based on the remaining filiform elements.

As will be clear to a person having ordinary skill in the art, the embodiments of the present invention can be used to locate blood vessels in different situations. For instance, the locator can locate blood vessels when taking blood samples from an arm of a patient or for gaining access to a femoral or jugular vein. Although it can be used on any kind of patients it is especially advantageous for elderly people and children since it does not lead to discomfort and has high sensitivity. Additionally, one or more of the embodiments may be implemented in a cost effective way and does not necessarily require complicated equipment. One or more embodiments provide a way to sense a blood vessel without the use of traditional needles, which may cause injury to tissue or the blood vessels themselves. Moreover, the time required to locate a blood vessel is significantly reduced since multiple filiform elements can be introduced in parallel compared to using trial and error multiple times using a single needle.

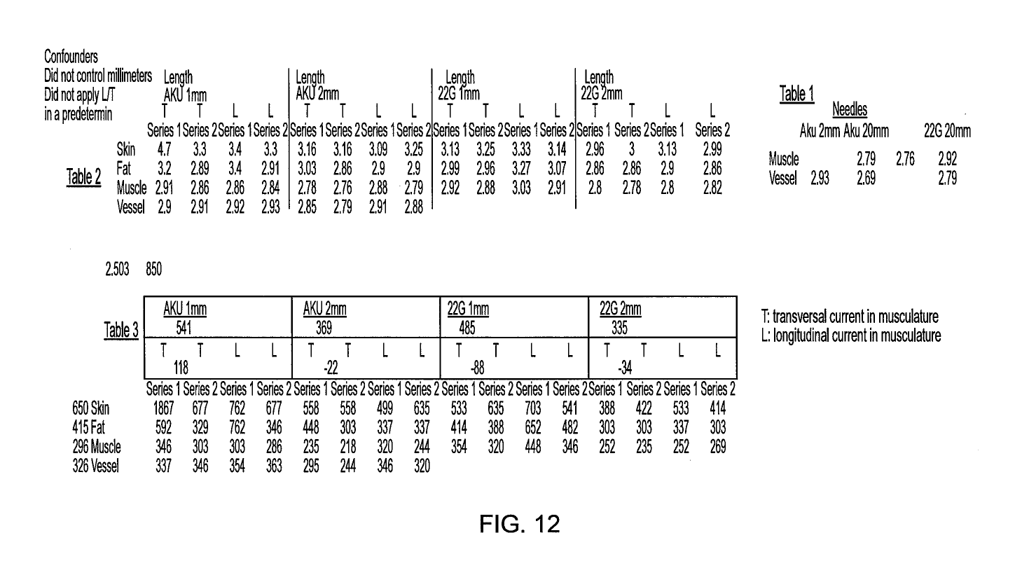

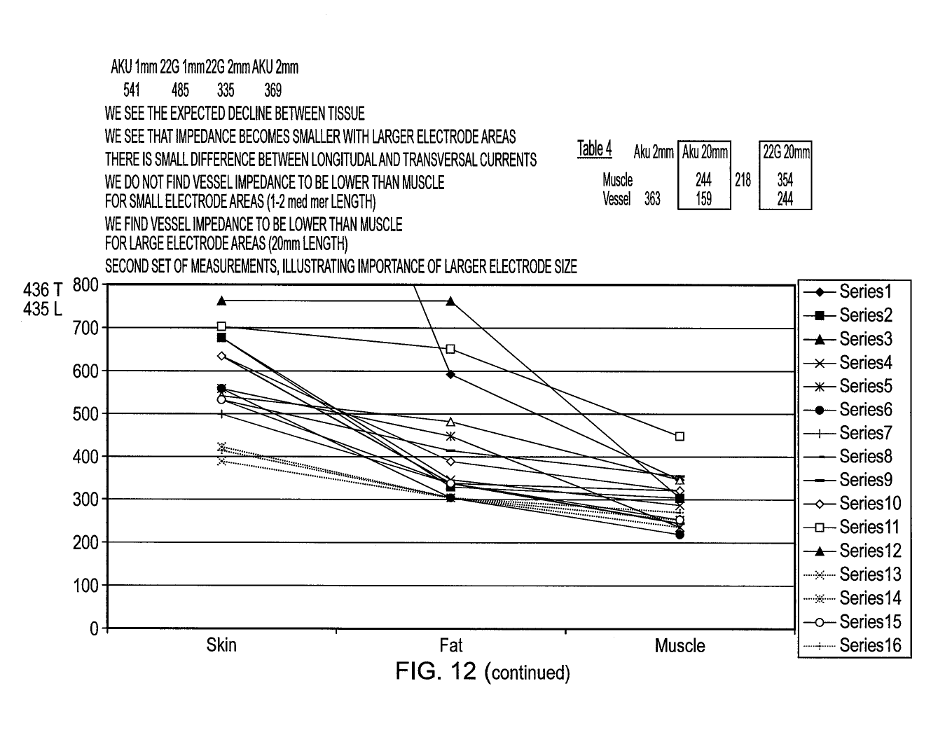

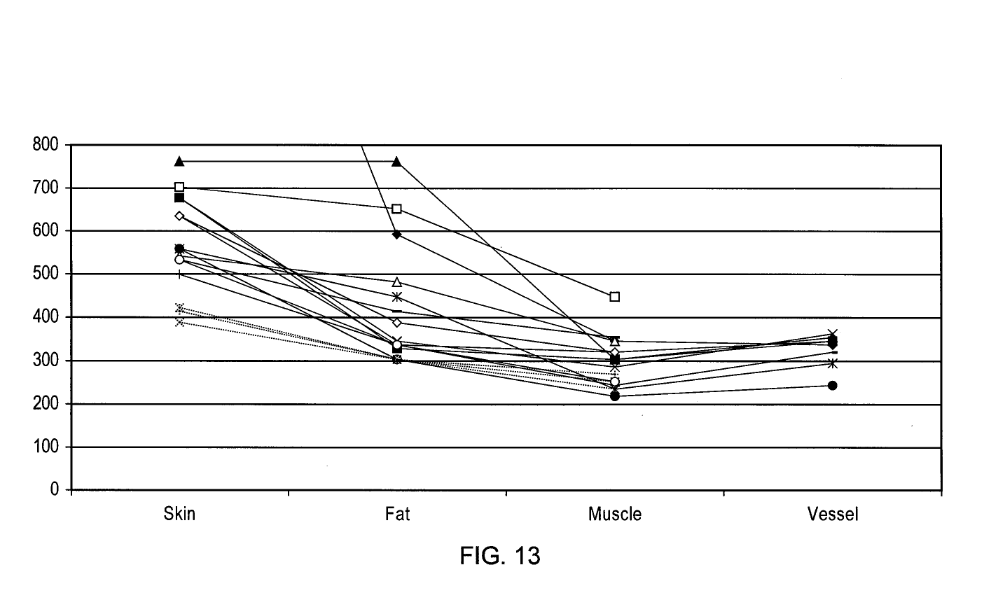

FIGS. 12 and 13 illustrate results from an animal experiment, where an impedance measurement system (running at 32 kHz) measured the impedance using varying sized needle electrodes in different kinds of tissue. A second electrode, which was a defibrillator electrode, was placed on the skin of the animal. Different types of needles were used as stated in Table 1, where AKU denotes acupuncture needles while 22 G denotes a thicker 22 Gauge needle. FIGS. 12 and 13 illustrate that there is no useful difference in impedance between muscle tissue and blood when needle electrodes have a length of 1-2 mm. Table 2 lists the voltages measured and Table 3 lists the corresponding impedance values in ohms. Table 4 lists the result of a second experiment, where now the needle electrodes had a length of about 20 mm. With this experiment, impedance in the blood vessels was found to be much smaller than the impedance in muscle tissue. Based on this experiment, in one embodiment an electrode length of about 20 mm is preferable if the impedance measurement system is running at 32 kHz. However, because blood and tissue impedance vary with frequency, a preferred impedance measurement system uses several frequencies to measure the impedance characteristics. In one embodiment, both the imaginary component and the resistive component of the measured signal are used as tissue and can be represented by both resistive and capacitive components. Consequently, needle electrode length becomes a function of sensitivity and specificity of tissue discrimination by an impedance measurement system. A preferred solution is optimized to detect those vessels that are the target vessels, and to not detect smaller vessels that might form a branch of the larger vessel. Hence, in one embodiment a needle electrode length of 15-20 mm is used if the target vessel diameter is 10-15 mm. This assumes that the needle electrode and/or filiform element is entered with an angle of 30-45 degrees.

In order to control cross contamination, in some embodiments the filiform elements are arranged in a cartridge, where the cartridge is disposable and where the cartridge may hold the whole sensor system or part of the sensor system. The cartridge may further be arranged with an appropriate release mechanism such that just those filiform elements that are in position in a vessel remain, and all other elements are retracted, such as shown in FIG. 11B. In one embodiment, the release is controlled by a microprocessor according to sensor outputs. In another embodiment, the release is controlled by an operator according to display readouts.

The filiform element may be introduced to the tissue manually or automatically. When the filiform element(s) are introduced to the tissue automatically, one or several motors may be arranged to feed the filiform elements through the tissue. The feeding is controlled by a microprocessor, which will stop feeding the filiform elements when the sensor output indicates that the blood vessel has been found or when the filiform element has traveled a predetermined distance. In some embodiments, the microprocessor advances the filiform elements in a non-uniform fashion.

Needles estimated to have the highest chance of accurate placement are advanced preferentially to limit injuries, both real and perceived. In other embodiments signals from several sensors can be combined to provide an indication of blood vessel location. One possible way of combining the signals comprises providing an indication for the sensors where the signal amplitude exceeds particular predefined value. The predefined value can be established as a priority or by a means of the detected signals, such as related to geometric positions providing a map of the area. A comparison with a given threshold can also be provided for the case where a single filiform element is used.

To ensure stability when progressing the needles and/or filiform elements, embodiments of the invention may also include a base plate with adhesive properties applied to the skin in order to prevent or limit relative movement between the skin and the filiform elements and/or needles. Such adhesive properties can be provided by use of adhesives or by use of a moderate suction. With suction, the base plate is arranged for instance with a set of holes each connected to a central vacuum reservoir or pump.

Although many different features of the invention have been described individually or in combination with one particular embodiment, as will be clear to a person having ordinary skill in the art, it is fully possible to combine different features of each embodiment to provide a locator according to the invention. For instance, it is thus possible to provide a locator with a holder, a light emitting device in the display and releasing mechanism for the filiform elements.

Although the present invention has been described with reference to the disclosed embodiments, persons skilled in the art will recognize that changes may be made in form and detail without departing from the spirit and scope of the invention. Such modifications are well within the skill of those ordinarily skilled in the art. Accordingly, the invention is not limited except as by the appended claims.

What is claimed is:

1. A blood vessel locator, comprising:
at least one filiform element;
at least one sensor device coupled to the at least one filiform element, respectively, the sensor device operable to detect a parameter indicative of a presence of a blood vessel; and
a display coupled to the at least one sensor device, the display device operable to provide an indication of the presence of the blood vessel.

2. The blood locator according to claim 1, wherein the at least one filiform device is an acupuncture needle, an electrode or a thin wire.

3. The blood locator according to claim 1, wherein the sensor device is an impedance measuring device, a blood sensor, an acceleration sensor, a pressure sensor, a flow sensor, a temperature sensor, or any combination thereof.

4. The blood locator according to claim 1, wherein the display is a light source, a sound emitting device, a tactile feedback device or any combination thereof.

5. The blood locator according to claim 1, further comprising a sleeve arranged for being placed around the filiform element to provide rigidity.

6. The blood locator according to claim 1, wherein the filiform element is arranged for use as a guide wire for a hollow device.

7. The blood locator according to claim 1, further comprising a processor for to control activation of the display based on signals from the sensor device.

8. The blood locator according to claim 7, wherein the processor is operable to analyze sensor signals to extract identification parameters, compare the identification parameters with threshold values, and based on the results of the comparison provide a signal to the display.

9. The blood locator according to claim 1, further comprising a plurality of filiform elements coupled to a holder.

10. The blood locator according to claim 9, wherein the filiform elements are releasably coupled to the holder.

11. The blood locator according to claim 9, wherein the individual filiform elements are coupled to a respective sensor.

12. The blood locator according to claim 9, wherein the plurality of filiform elements are arranged in an array of filiform elements.

13. Method for locating a blood vessel in a body, comprising:
providing a sensor device into the body;
detecting a parameter indicative of a presence of a blood vessel; and
indicating a presence of a blood vessel location.

14. The method according to claim 13 wherein the act of providing an indication of a blood vessel location is provided by a visual or audio element.

15. The method according to claim 13 wherein the act of providing a sensor device into a body comprises providing a filiform element coupled to the sensor device into the body.

16. The method according to claim 15 wherein the act of providing a filiform element coupled to the sensor into the body comprises providing a plurality of filiform elements coupled to a respective sensor device into the body.

17. The method according to claim 13 wherein the sensor device comprises an impedance measuring device, a pressure sensor, a blood sensor, an acceleration sensor, a flow sensor, or a temperature sensor.

18. The method according to claim 13 wherein the act of providing a sensor device into the body comprises providing a plurality of sensor devices into the body.

19. The method according to claim 13 wherein the act of detecting a parameter indicative of a presence of a blood vessel comprises detecting the parameter without penetrating the blood vessel.
